(19) United States
(12) Patent Application Publication
(10) Pub. No.: US 2025/0205086 A1
(43) Pub. Date: Jun. 26, 2025

(54) METHOD FOR DETERMINING IRRADIATION PARAMETERS AND APPARATUS FOR IRRADIATION

(71) Applicant: OD-OS MacuTherm GmbH, Teltow (DE)

(72) Inventors: Arnd Rose, Stahnsdorf (DE); Kay-Uwe Amthor, Teltow (DE); Eberhard SCHMIDT, Kleinmachnow (DE)

(21) Appl. No.: 18/851,044

(22) PCT Filed: Mar. 24, 2023
(86) PCT No.: PCT/EP2023/057719

§ 371 Date: Sep. 25, 2024

(30) Foreign Application Priority Data
Mar. 25, 2022 (EP) 22164545.0

(51) Int. Cl. A61F 9/008

(52) U.S. Cl. CPC A61F 9/00821; A61F 2009/00851; A61F 2009/00857; A61F 2009/00863

(57) ABSTRACT

The invention relates to a method for determining irradiation parameters for the operation of a laser (2) for irradiating one or more target regions of the retina (16) of an eye (4) by means of an irradiation beam (11), in which method the retina is fully or partly illuminated by an illumination device (5, 5′) under defined illumination parameters, and, during and/or shortly after the illumination, at least one recording, in particular at least one camera image, is captured of at least one target region of the retina, and in which method at least starting values of the irradiation parameters are determined for the irradiation of one or more target regions of the retina using the recording(s) or camera image(s). The invention also relates to a corresponding apparatus in the form of an expanded ophthalmoscope and allows, with the lowest possible complexity, objectified determination of starting values for irradiation parameters during laser treatment of a retina.

The invention is in the field of medical measurement technology and relates specifically to a method for determining irradiation parameters for irradiating target regions of the retina of an eye.

Methods are known for the treatment of various eye diseases in which laser treatment of the retina can bring about a change in tissue through so-called laser coagulation. For all types of tissue treatment using a laser, it is important to find out as much as possible in advance about the interaction of the laser beam used with the tissue of the individual patient.

For this purpose, it is known from the prior art to use a laser to perform so-called titration shots, i.e. to expose certain tissue regions of the retina to a laser beam under certain conditions in order to experimentally determine the effect of a certain intensity of laser treatment on the tissue. The disadvantage of this method is that, on the one hand, certain regions of the retina are exposed without sufficient prior knowledge of the stress caused by laser treatment and, on the other hand, a certain amount of time is required to evaluate such experiments, as the reaction of the tissue to the experimental laser treatment must be awaited. Although this only takes a few seconds for each titration shot, it is common to use a number of titration shots, especially with variable intensity, to determine the response of the tissue to different laser intensities. The effect of the titration can also be determined by other examination methods if it remains below the visibility threshold in the color image.

For example, a method is known from WO 2011/038935 A1 in which, in connection with titration shots on regions of a retina, images of the retina are captured by means of a camera, from which a discoloration of the tissue regions hit by the laser can be detected. In addition, a difference image is generated between camera images taken before and after the titration, so that the effect of the titration can be derived from the difference values.

Against the background of the prior art, the present invention is based on the object of creating a method and an apparatus for determining irradiation parameters for the operation of a laser for irradiating one or more target regions of the retina of an eye, with which the irradiation parameters can be determined as reliably as possible with the least possible loss of time and the least possible complexity.

The object is solved according to the invention with a method having the features of claim 1.

According to this, the invention relates to a method for determining irradiation parameters for the operation of a laser for irradiating one or more target regions of the retina of an eye by means of an irradiation beam, in which method the retina is fully or partly illuminated by an illumination device under defined illumination parameters, and, during and/or shortly after the illumination, at least one optical recording, in particular at least one camera image, is captured of at least one target region of the retina, and in which method at least starting values of the irradiation parameters are determined for the irradiation of one or more target regions of the retina using the recording(s) or camera image(s).

The aim of the method is to make it possible to determine the irradiation parameters for the operation of a laser as quickly, automatically and reproducibly as possible prior to treatment. According to the prior art, it is often common practice to experimentally apply laser shots (titration shots) to regions of the retina in order to determine the reaction of the tissue in the individual patient. The surgeon then often assesses the degree of tissue discoloration as a result of the laser effect according to his personal impression and adjusts the parameters of the laser for irradiation during the actual treatment.

The method according to the invention makes it possible to completely dispense with or significantly reduce trial operation of the laser prior to treatment. By using recordings or camera images of the retina obtained in real time, an assessment of the individual retinal tissue is objectified, transparent, comprehensible, reproducible and verifiable.

With the method according to the invention, it may be sufficient to finally determine the irradiation parameters for the subsequent operation of the laser for irradiation in the manner described, so that the retina must be exposed to the laser radiation exclusively for treatment and for the experimental determination of the necessary laser intensity in the context of a titration process.

The illumination device can be a source of visible light, for example a light-emitting diode or a laser, for example the treatment laser itself, surface illumination, for example by means of an illuminated reflective surface, a frosted glass pane illuminated from the rear or a light-emitting diode matrix or another broadband light source. Illumination beyond the visible regions of the optical spectrum in the infrared or ultraviolet range can also be considered. In this context, the defined illumination parameters can be understood to mean, for example, a luminous intensity and the wavelength or a wavelength distribution of the emitted radiation. These can be set to specific values or characteristics, for example. The illumination parameters, such as the illuminance, distribution of the illumination intensity and the spectrum/wavelength distribution, can also be measured alternatively or additionally and the measured values can then be used when evaluating the recordings. It should also be noted that radiation in the non-visible range, such as in the infrared range, can also be used, wherein a captured recording or camera image in this case is also an infrared image that is recorded with corresponding detectors. For example, hyperspectral sensors can also be used for the recordings. Certain regions of the retina, such as target regions, can also be selected automatically or manually for illumination. During automatic selection, certain geometric lighting patterns can be specified and selected. The method can also use a rastering/scanning method of illumination and scanning, in which, for example, the target regions or the entire retina are illuminated with a focused light source that only covers a narrowly defined region and the reflection is detected by means of a light-sensitive, non-location-resolving sensor, such as a photodiode. In this way, the entire region of the retina can be optically scanned continuously and an optical image of the reflectivity and/or absorption intensity can be created. Conversely, the entire retina can also be illuminated simultaneously and the light reflected from a limited region of the retina can be detected in all regions of the retina or in the target regions by means of a spatially resolving optical sensor, thus creating an optical image of the reflectivity and/or absorption strength of the retina.

In the context of the present invention, an optical recording can also be understood to mean, for example, an optical imaging recording of the retina.

As imaging methods for capturing a recording or a camera image of the retina, for example, the methods of fundus photography, fundus reflectometry, autofluorescence imaging, photoacoustic imaging and functional optical coherence tomography with corresponding illumination devices and illumination methods can also be used. These methods can be used, for example, to determine the pigmentation intensity of the retina, which is decisive for the absorption strength of the irradiation beam used later. However, the pigmentation intensity can also be generally determined from the brightness of the retina, which is recorded with the image or the camera image.

Often only certain individual or several target regions of the retina are to be treated with a laser beam. In this case, it may be sufficient to illuminate the target regions and capture a recording or a camera image of the target regions. However, it may also be useful to capture recordings/camera images of the entire retina, as the necessary irradiation intensity of the laser during treatment can also be estimated in other regions of the retina not intended for treatment, taking into account the pigmentation intensity. Other, additional non-local characteristics of the person to be treated can also be taken into account when determining the necessary radiation intensity. Further information on this topic is provided below.

A particular implementation of the method described above may also provide that the intensity of the pigmentation for one or more target regions of the retina and/or the intensity distribution of the pigmentation on the retina are determined by means of the recording(s) or the camera image(s) in order to determine the starting values of the irradiation parameters.

It may also be provided that, in order to determine the starting values of the Irradiation parameters for one or more target regions of the retina, additional correction values are determined with regard to the expected absorption and/or scattering of the irradiation beam in the vitreous body of the eye on the way to the target regions to be irradiated, wherein the correction values are determined in particular from the optical quality of the optical recording, in particular from an image sharpness or an image contrast.

The sharpness or contrast of the image can be assessed on the basis of imaged inhomogeneities of the retina, such as blood vessels or nerve nodes. The sharpness/contrast information obtained from different regions of the retina can be compared with each other and/or with stored reference images in order to assess the influence of scattering/absorption in the vitreous body of the eye on the image sharpness on the way to the retinal region in question and to determine this parameter in general for an eye or also depending on the location on the retina.

For example, it may be advantageous to detect the melanin pigmentation of the retina and, for this purpose, to irradiate the retina with light in the visible range with wavelengths or even exclusively with wavelengths that are particularly well absorbed by the melanin pigmentation.

Wavelengths of illumination and/or detection in the range between 450 nm and 1064 nm can be advantageous for a measurement. In particular, measurements at otherwise 514 nm, 532 nm or 577 nm can be advantageous. The scattering and absorption of radiation by melanin is strong in this region. In addition, the wavelengths at which the differences between the absorption strengths of melanin and blood are significant can also be advantageous. It may be useful to compare recordings taken at a wavelength of 480 nm (local minimum absorption in blood) with recordings taken at 550 nm (local maximum absorption in blood). Reference values for reflectance measurement can be, for example, the papilla as a nerve node, which shows a strong reflection in a healthy state, or regions with blood vessels in which the presence of oxygen-rich or oxygen-poor blood can be selectively detected and useful comparison values can be generated using suitable wavelengths that can detect these blood variants.

During the subsequent laser treatment of the retina, the irradiation beam is partially absorbed or scattered due to the fact that the vitreous body of the eye is not 100% transparent. This effect should be taken into account when determining the irradiation parameters for the laser, so that the laser intensity is selected slightly higher than is directly necessary for the treatment of the retina in order to compensate. The absorption of the irradiation beam depends on the individual characteristics of the vitreous body of the patient to be treated. In the method according to the invention, the absorption can be estimated, since the illuminance with which the retina is illuminated is known and the intensity of the backscattered signal, for example the reflected light, depends both on the pigmentation intensity of the retina and on the absorption of the light in the vitreous body. In order to be able to separate the two effects influencing the backscattering, several measurements can be carried out under different optical conditions. Further details are provided below.

In many cases, it may be useful for the retina to be illuminated with radiation with a known wavelength distribution, which in particular comprises infrared radiation and/or light in a wavelength range absorbed by melanin, in order to generate a camera image, wherein in addition the luminous intensity of the illumination device and/or the total light power incident on a unit region of the retina and/or the light power incident on a unit region of the retina in a defined wavelength range is determined and wherein the brightness values and/or colors of the camera image are used to determine the starting values of the parameters for the irradiation of the retina.

From empirical values for the necessary intensity of the irradiation beam in connection with previously measured brightness values of the retina, starting values of the irradiation parameters can be determined for the individual case, wherein, for example, the absorption of the irradiation beam by the vitreous body can be represented by an assumed constant value, which may depend, for example, on the age of the patient.

In addition, it may advantageously be provided that the thickness of the retina is determined for one or more target regions and taken into account when determining the starting values of the irradiation parameters, with the thickness of the retina being determined in particular by creating an OCT thickness map of the retina, and/or that the type and intensity of the pigmentation of different regions of the body other than the retina of the person to be treated, in particular the type and intensity of the pigmentation of the iris of the eye, or of the skin and of the hair, are used to determine the starting values of the irradiation parameters.

Using an OCT measurement (optical coherence tomography), the thickness of the retina can be determined depending on the location, for example to detect edema in the retina, which often absorbs part of the laser radiation itself, so that certain parts of the irradiation beam do not reach the pigment layer during the actual treatment. The OCT thickness map can therefore be used to determine a location-dependent correction variable for the irradiation parameters over the entire area or partial areas of the retina.

In another implementation of the described method, it may also be provided that, in addition to the captured recording(s) or camera images, one or more specified or specifiable reference images or reference recordings are used to determine the starting values of the irradiation parameters.

The reference recordings or reference images can, for example, represent target states of the retina, which can be compared with the captured images/camera images in order to perform a difference value analysis and determine a necessary irradiation intensity or the necessary irradiation parameters or their starting values from the differences between the captured image and the reference image. The irradiation parameters that determine the treatment intensity or irradiation intensity can typically include the strength of the laser beam, i.e. its energy, and its cross-sectional region in the impact region/target region or the size of the laser beam spot as well as the duration and number of laser pulses emitted and pauses between the laser pulses. Finally, reference recordings can also be taken from databases, for example, or obtained from previously recorded recordings/camera images of the same patient.

In a further implementation of the method described above, it may also be provided that, in order to determine the irradiation parameters or at least the starting values of the irradiation parameters for one or more target regions of the retina to be irradiated, on the one hand the intensity of the pigmentation of the target region(s) and on the other hand the expected absorption of the irradiation beam in the eye lens on the way to the target regions are determined independently of each other by carrying out at least two measurements under different illumination conditions, at least one of the following parameters in particular being varied in the two or more measurements: size of the focused light spot on the retina, wherein in each case the deviation from an expected light intensity distribution on the area of the focus spot is measured, direction of incidence of an illumination beam of the illumination device through the pupil and the vitreous body for illumination of the respective target region, direction of incidence to the target region and/or direction of emission of a detected reflected illumination beam of the illumination device, wavelength range or wavelength distribution of an illumination beam of the illumination device.

This method makes it possible to determine the distribution of the pigmentation intensity on the retina on the one hand and the absorption of a laser beam by the vitreous body as a function of the laser target point on the retina and the light path on the other, independently of each other. For such a measurement, for example, the light path when illuminating a target region of the retina can be changed for various successive individual measurements, but the same target region can be illuminated and recorded in each case. On the other hand, a target region of the retina can also be illuminated by the illumination device in different ways in several measurements while the light path remains the same, so that the influence of the reflectivity of the retina can be varied by illumination with different spectra or by light in different wavelength ranges.

Alternatively or additionally, it may also be provided that, in order to determine the irradiation parameters for one or more target regions of the retina to be irradiated, the intensity of the pigmentation of the target region(s) and/or the expected absorption of the irradiation beam in the lens of the eye on the way to the target regions are determined on the one hand by carrying out at least two measurements, wherein of the measurements is directed to the absorption and/or reflection characteristics in the region of the papilla and/or at least one blood vessel or part of a blood vessel in the retina, and the other being directed to the absorption and/or reflection characteristics in the region of the papilla and/or at least one blood vessel or part of a blood vessel in the retina, while a second measurement is directed to the absorption and/or reflection characteristics in one of the other regions of the retina to be irradiated.

The method described can also provide that, in order to determine the irradiation parameters for one or more target regions of the retina to be irradiated, at least two locations of the retina are each illuminated successively or simultaneously with different illumination characteristics, in particular different illumination intensities, and the intensity of the reflected signal is recorded in each case, that the functional relationship between the illumination characteristic, in particular the illuminance, and the intensity of the reflected signal is determined for the locations measured in this way in each case, and that the slope of the functional curve is determined by comparing the functional relationships at the measured locations, in particular the slopes of the functional curves. the intensity of the reflected signal, in particular the slope of the function curve in the case of a linear relationship, is determined and that by comparing the functional relationships at the measured locations, in particular the slopes of the function curves, the ratio of the reflectivities at the measured locations is determined, wherein in particular one of the locations is located on the surface of a papilla or a blood vessel or at least partially encompassing the latter.

The optic nerve head (papilla) is usually not pigmented and reflects light much more strongly than the pigmented parts of the retina. In addition, reflectivity in the papilla region is only slightly dependent on the characteristics of an individual patient's retina. The papilla can therefore serve as a “reflection standard” or reference for a comparative measurement of the intensity of the pigmentation.

The veins of the retina can also be detected and used for a comparative measurement in the regions of the retina defined by them. The blood passing through has a certain different absorption characteristic than the melanin.

Absorption also depends on whether the blood is saturated with oxygen or not, i.e. which type of vessel is used. In order to be able to measure and evaluate these properties of absorption and reflectivity in the blood vessels in a differentiated manner, it can be useful to carry out a reflectivity or absorption measurement at wavelengths of the illumination light that are particularly strongly influenced by the oxygen-saturated light

or the deoxygenated blood. As an alternative to the corresponding selection of the illumination light, the corresponding meaningful wavelengths can also be selected on the detection side.

It has been found that the functional relationship between an illumination characteristic, in particular the illuminance on the one hand and the intensity of the reflected radiation on the other hand at a location on the retina allows a good determination of a value that represents the reflectivity of the retina at this location. In the case of a linear relationship between the illuminance and the intensity of the reflected radiation, the linear regression method can be used, for example, to determine a gradient of the straight line that forms the function graph for a number of measurements in order to compensate for measurement errors. The slopes of the straight lines, which can then be assigned to the respective measured locations of the retina, can form a relative measure of reflectivity. The reflectivity values assigned to the measurement locations can then be calibrated by comparison with a corresponding correlation, in particular a straight line gradient, at a location at which the reflectivity is known or can be well estimated. The reflectivity at one location on the retina can then be used to determine the absorption intensity for a irradiation beam.

The wavelength of the radiation illuminating the respective measuring location can also serve as a variable illumination characteristic, so that two locations are then illuminated one after the other with radiation of different wavelengths, the intensity of the reflected radiation is measured in each case and the functional relationship is determined and compared for the two locations.

The differentiation of reflectivity at different wavelengths can be achieved either by successive illumination at different wavelengths with simultaneous broadband detection (or detection adapted by a wavelength window) or with broadband illumination covering a continuous spectrum or with illumination by a laser array comprising a plurality of individual wavelengths. In the latter case of broadband excitation/irradiation, wavelength-selective detection of the reflected radiation is then provided, for example by defining color channels, using wavelength-selective filters or using a spectrometer or a multispectral camera.

Since the reflectivity in the region of the papilla varies only slightly, a deviation of the determined characteristic and the reflectivity value determined from it in a first step from a reference value can be attributed to the influence of absorption and/or scattering of the radiation on the measurement path through the eye of the individual patient.

Deviations of the measured values from the reference value of the reflectivity at a reference location such as the papilla or the blood vessels can thus be used to determine the attenuation due to absorption and/or scattering in the body of the eye.

By taking a number of measurements at different locations on the retina, the influence of the pigmentation on the captured recording or the captured camera image on the one hand and the influence of the absorption of the vitreous body on the other can be determined independently of each other in an evaluation and taken into account when determining the irradiation parameters. It should also be noted that the direction of incidence and the direction of emission of the illumination beam also refer to the light path that the laser beam travels through the vitreous body.

The type of linking of illumination parameters and the detected variables/signals of the respective captured recording or the respective captured camera image on the one hand and the irradiation parameters for the operation of the laser on the other hand can take a variety of forms. In the simplest case, this can be a mapping table, which can exist as a multidimensional matrix and which can be stored, for example, in a computer or a non-local or distributed data processing system. The link can also be in the form of an algorithm or a neural network, which can be trained using self-learning or guided learning methods, for example.

Against this background, the present invention can also relate to a method for training an algorithm or a neural network for determining the starting values of irradiation parameters according to the method described above, wherein, for a plurality of individual treatments, datasets of target regions to be irradiated determined from images and the associated illumination parameters and, in particular, secondary information additionally acquired in each case as input information, and, on the other hand, the irradiation parameters set during the respective subsequent laser treatment as result variables are linked together as training data.

The training data can, for example, initially be obtained by parallel operation of a conventional method and the method described here, wherein, as described above, a recording or a camera image is captured under defined illumination conditions, and the necessary treatment intensity, i.e. the intensity of the treatment laser beam, e.g. represented by laser power, pulse duration, pulse number and repetition rate, is determined in a conventional manner using titration methods. This can then be linked to the previously recorded recordings/camera images for the target regions of the retina that are to be treated, so that the learning system is trained with this data.

The individual treatments used to train the algorithm can also be carried out according to the method of the invention described above, wherein the starting values for irradiation parameters are first determined and then the treatment is started and the success of the treatment is assessed by checking the discoloration of the target regions and, if necessary, the irradiation parameters are corrected. By entering the correction values or linking the correction values with the illuminance parameters and the initially determined starting values for the irradiation parameters, the algorithm can be further trained for improvement.

Additional secondary information can be entered as variables that prove to be influential in determining the irradiation parameters. Such secondary information can be, for example, the patient's age and certain parameters of their state of health as well as specific parameters of the patient's retina to be treated. For example, a measured integral pigmentation intensity of the skin or the iris of the eye or a measured pigmentation intensity of the hair of the head, the hair of the eyebrows or the skin of the patient as well as the color of the skin pigmentation, the color of the iris and the color of the hair can be taken into account.

When determining the starting values of the irradiation parameters for all target regions, it is also possible, for example, to carry out a calibration with one or more of the following calibration parameters of the person to be treated: measured integral pigmentation intensity of the skin, measured integral pigmentation intensity of the iris of the eye, measured pigmentation intensity of the hair (hair, eyebrows), color of the skin pigmentation, color of the iris, color of the hair. Such a calibration can also provide, for example, that the listed variables are not included in the algorithm for determining the irradiation parameters, but that after determining an irradiation intensity, the results are calibrated with the specified variables, for example by multiplying the irradiation intensity, given by the laser energy and the length of the laser pulse, by a specific calibration factor. t is determined, the results are calibrated with the stated variables, for example by multiplying the irradiation intensity, given by the laser energy and the length of the laser pulse, by a specific calibration factor.

The invention can also relate to a method for determining irradiation parameters for the operation of a laser for irradiating one or more target regions of the retina of an eye with a irradiation beam, in which first of all starting values of the irradiation parameters are determined according to the method described above, in which then at least one target region is irradiated with a titration beam or a irradiation beam with the starting parameters, in which at least one continuation recording or a continuation camera image of the target region or regions of the retina irradiated with the irradiation beam is then acquired and in which, using the continuation recording(s) and/or the continuation camera image(s) and the starting values of the irradiation parameters, at least continuation values of the irradiation parameters for the irradiation of one or more target regions of the retina are determined.

In this way, it is possible to check the initially determined starting values of the irradiation parameters as soon as possible after their application for the success achieved and to adjust them if necessary or to determine continuation irradiation parameters from these. The corresponding corrections can also be used, for example, to provide the algorithm with further training data and thus improve it.

Finally, the invention also relates, in addition to the method described above, to an apparatus for irradiating one or more target regions of the retina of an eye with a irradiation beam, having a laser for generating the irradiation beam, an illumination device for illuminating at least one or more target regions of the retina with defined illumination parameters and with an optical recording device, in particular a camera, for capturing at least one recording or an image of at least one target region of the retina and with a processing device which is configured to determine at least starting values of the irradiation parameters for irradiating one or more target regions of the retina with a irradiation beam using the illumination parameters and the optical recording(s) or camera image(s).

This apparatus can be used to carry out the various implementations of the method described above. The processing device can be designed as a data processing device that contains an algorithm that assigns the starting values for irradiation parameters to the illumination parameters and the variables recorded with the recording or the camera image. This algorithm can either be fixed or trained with training data as part of a learning system with artificial intelligence, as described above. The processing device may also include, for example, a neural network capable of learning in the manner described by forming its links through the training process.

The aforementioned apparatus can, for example, be completely independent of the treatment device with the treatment laser or also be connected to the treatment device or integrated into it. For example, the apparatus according to the invention can also be partially centralized, wherein the processing device can be arranged non-locally in a server or a remote data processing system of another type and receives the determined data by means of a suitable communication method. In this way, the processing device can be fed and trained with measurement data from many different locations, and it can control local treatment devices remotely. In this way, optimal training of the processing device with actual data from one or more patients can be ensured.

In the following, the invention is shown in embodiments with the aid of figures and explained below. In the figures:

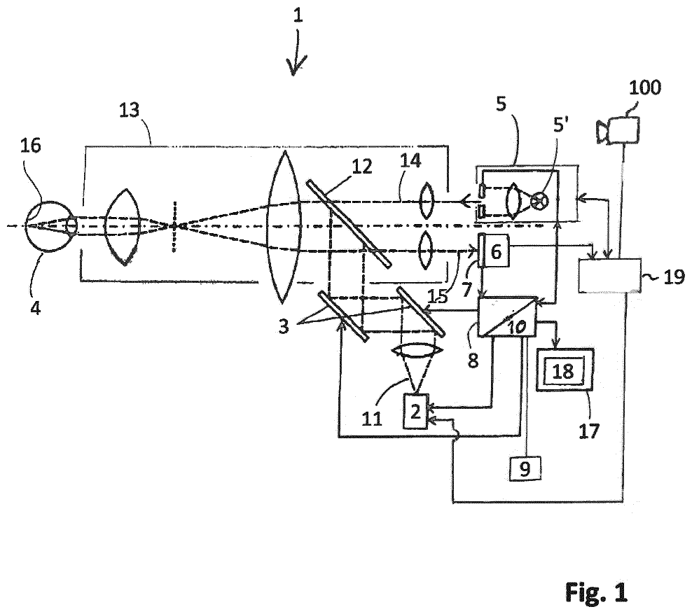

FIG. 1 shows an ophthalmoscope with an apparatus for irradiating the retina of an eye,

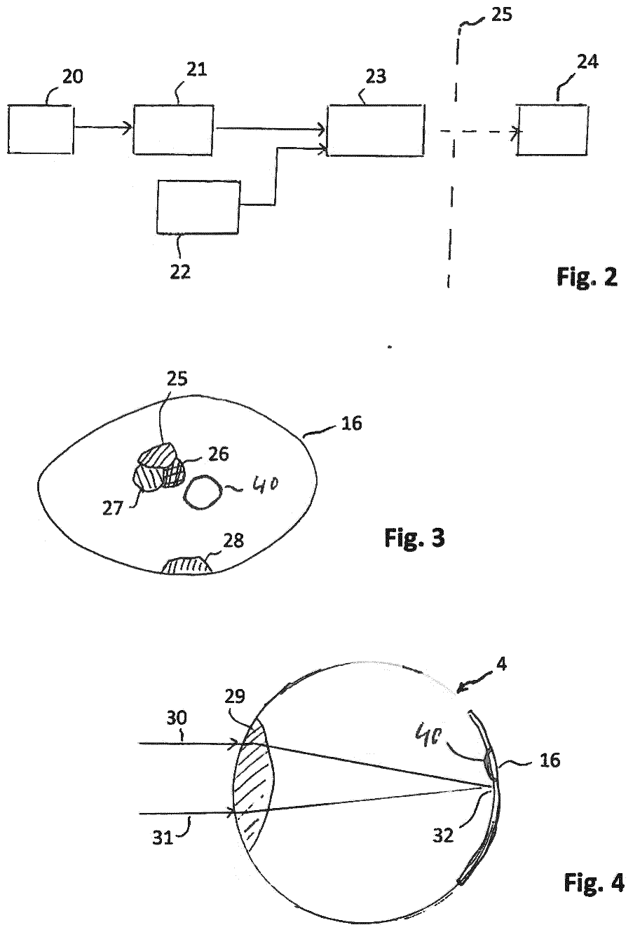

FIG. 2 shows a flow chart for a method for determining starting values for irradiation parameters,

FIG. 3 shows an image of target regions of a retina,

FIG. 4 shows an enlarged view of the light path through the vitreous body of an eye to the retina,

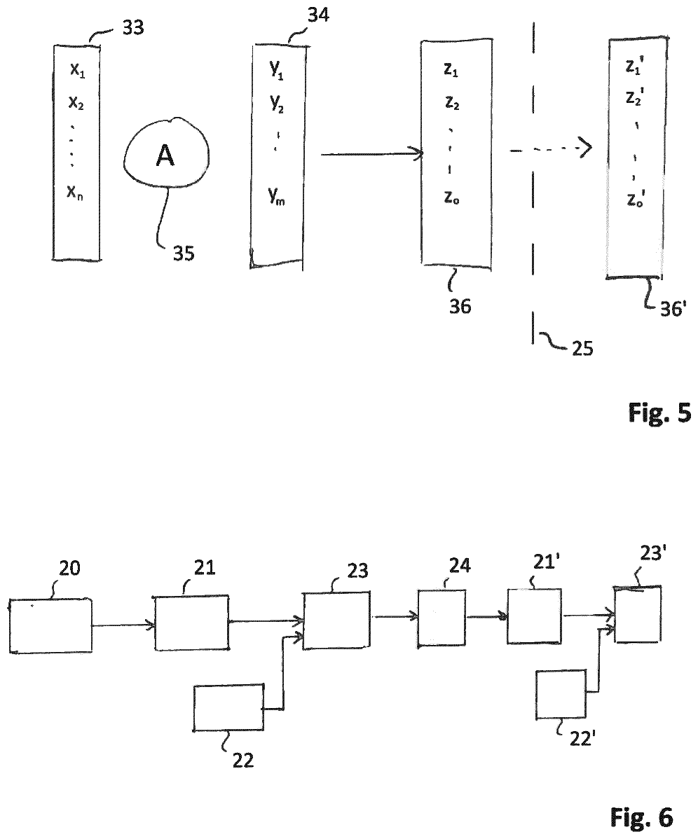

FIG. 5 shows an example of illumination parameters, measured values representing a recording of the target regions of a retina, and assigned starting values for irradiation parameters,

FIG. 6 shows a second flow chart of a method in which starting values for irradiation parameters are first determined and then checked,

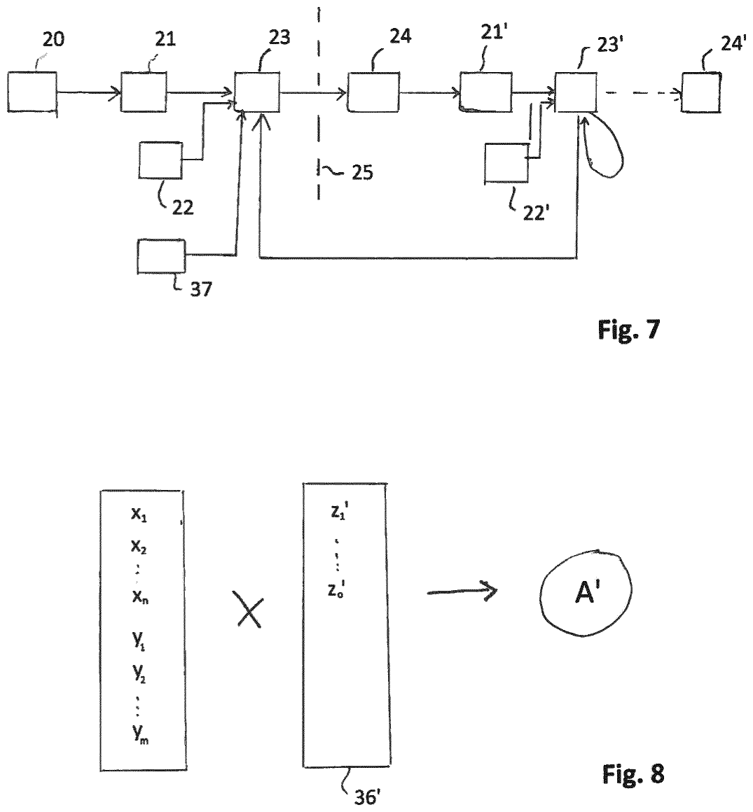

FIG. 7 shows a third flow chart, which illustrates a method for training an algorithm that is perfected in a self-learning process and assigns the starting values of irradiation parameters to the illumination parameters and other correction variables,

FIG. 8 schematically shows the linking of input variables and output variables for training the algorithm, and

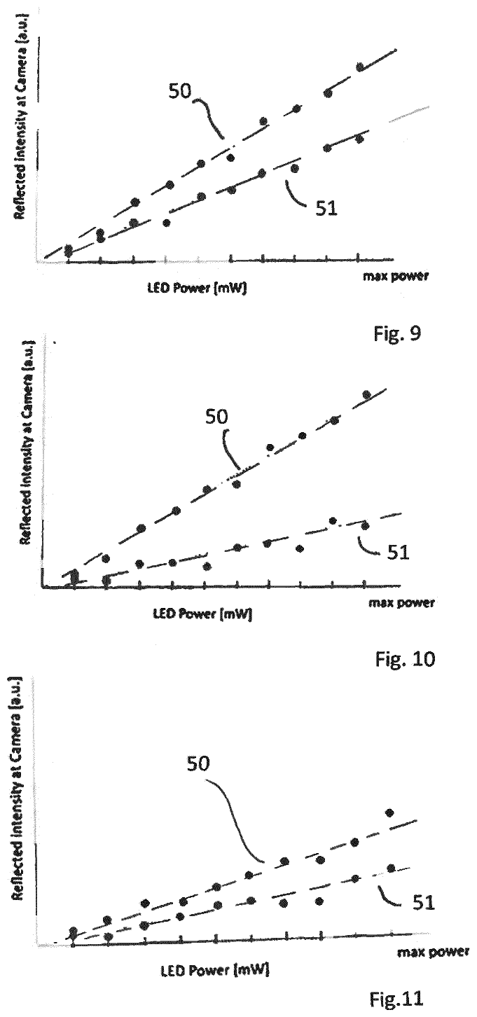

FIGS. 9, 10, 11 show the functional relationship between the illuminance and the intensity of the reflected radiation for the papilla/optic nerve and other regions of the retina for three different cases.

FIG. 1 shows an apparatus according to the invention or an apparatus for carrying out the method according to the invention in the form of an ophthalmoscope or an apparatus that combines the functions of an ophthalmoscope with the functions of laser treatment of the retina.

The apparatus has an illumination device 5 with a radiation source 5′, which is configured to direct an illumination beam 14 onto the eye 4 and the retina 16 of a patient. This allows the retina 16 to be suitably illuminated for capturing a recording or a camera image. The illumination can, for example, be equipped with a light-emitting diode or an infrared diode as a light source or with a light source of another type that provides a defined wavelength spectrum. The light source can also be a UV light source, for example.

The ophthalmoscope also has a camera 6 with a sensor labeled 7. The sensor can be a CCD or CMOS sensor, for example. Instead of the camera 6, any other type of device can also be provided which is suitable, for example as a scanning device, for detecting radiation reflected or scattered by the retina.

The aim of operating the illumination device 5 and the camera 6 or an equivalent device is to obtain the most accurate, spatially resolved measurement data possible from the retina 16 under defined illumination conditions by recording an image of reflected radiation and thus to record or determine the properties of target regions to be treated.

The illumination beam 14 and the reflected radiation 15 are suitably collimated or focused by a suitable optical system 13 with mirrors and lenses in a manner known per se. The optical system 13 also has a beam splitter 12, which makes it possible to direct a laser beam from the treatment laser 2 onto the retina 16. Alternatively, the laser beam can also be coupled in without a beam splitter, for example by guiding it slightly offset to the side in relation to the illumination light. A control unit 8 can be provided to control the laser 2, which on the one hand controls the illumination device 5, for example by triggering it, and on the other hand captures a camera image from the camera 6 and directly controls the laser 2. The control unit 8 can also control deflecting mirrors 3, which direct the beam path of the irradiation beam 11 and thus enable the treatment of individual target regions on the retina 16.

For improved control of the laser 2, a processing device 19 is provided according to the present invention, which allows accurate processing of recordings/camera images from the camera 6 and links these to the known and defined parameters of the illumination of the retina 16.

By way of example, the ophthalmoscope also has a sensor 100, for example in the form of a camera, which enables the measurement of the pigmentation color and pigmentation intensity of the patient's skin, hair and/or iris.

An input device can also be provided with which such a parameter can be entered. In any case, these parameters are passed to the processing device 19 and taken into account there when determining the starting values for irradiation parameters.

In the past, the intensity of the laser treatment, i.e. the strength and/or duration of the laser pulses with which the laser 2 was operated, was carried out by an operator according to their own assessment after evaluating a recording of the retina. First, titration pulses were directed onto the retina and their effect assessed in order to scale the laser intensity.

By illuminating the retina with known illumination parameters and linking them to the recording of the retina, it is possible to assign an intensity of the laser treatment to these partially specified, partially measured values for each target region on the retina in an objectified manner by the processing device 19 of the laser treatment for each target region on the retina, wherein the intensity is given by the energy of the laser, the size of the laser spot on the retina and the number, repetition rate and duration of the pulse or pulses as well as the length of the pauses between the pulses. With the method according to the invention, at least starting values for such an irradiation by the irradiation beam 11 can be determined, with which the treatment of the retina can be carried out,

FIG. 2 shows a flow chart of the method according to the invention, in which in a first step 20 parameters of the illumination of the illumination device 5 are specified. However, the illumination parameters can also already be present and specified in the illumination device. Alternatively, it is possible for individual illumination parameters to be specified by the processing device 19, for example, and sent to the illumination device 5. The illumination parameters, such as the illuminance, distribution of the lighting intensity and the spectrum/wavelength distribution can also be measured and the measured values can then be used in the evaluation.

In a second step 21, the illumination device 5 or the light source 5′ is then operated and the retina is illuminated continuously or in pulses. At the same time, the reflected radiation can be recorded by a camera 6 in a further step 22. The step 22 can be carried out simultaneously with the illumination 21 or also with a time delay relative to the illumination if, for example, fluorescent radiation is recorded which is known to luminesce after the excitation radiation.

In a step 23, the illumination parameters and the measured values recorded by a camera are then linked together in a processing device 19 by an assignment function, for example an assignment algorithm, and starting values for the irradiation parameters, which are to be converted into irradiation by the operation of the laser 2, are determined from these variables by the linking. Only after the starting values have been determined can treatment 24 begin, which represents a further step. Step 24 of the actual treatment is separated from the determination of the starting values of the irradiation parameters by the dashed line 25 in the illustration.

FIG. 3 shows a schematic image of the retina 16 of an eye with different target regions. A target region 28 at the edge of the retina is shown, which is not as important for the actual visual performance of the eye as the target regions 25, 26, 27 in the center of the retina 16. The different shading of the individual target regions is intended to represent different pigmentation intensities or other varying parameters of the individual target regions. This should make it clear that the different target regions for treatment by laser 2 require different irradiation parameters. The size of the target regions 25, 26, 27, 28 can vary and can also be as small as a spatially resolved treatment by a laser 2 allows. FIGS. 3 and 4 also show the papilla and are labeled 40. The region of the papilla remains untreated and can serve as a reference for reflectivity compared to the melanin-bearing regions of the retina.

FIG. 4 shows a cross-section through the vitreous body of an eye 4 with an eye lens 29 and a retina 16. A point or target region 32 is indicated on the retina, which can alternatively be specifically illuminated with two different illumination beams 30, 31. Depending on where the illumination beam 30, 31 enters the lens of the eye, the light beam takes different paths through the lens and the vitreous body to point 32. If the measured values of the reflected light recorded by the camera 6 are compared with each other in terms of their intensity, the scattering or attenuation of the light on the various light paths through the vitreous body can be recorded in a differentiated manner. By varying the illumination beams, the entire vitreous body of the eye 4 can be gradually measured so that the influence of the vitreous body through attenuation and scattering on the illumination of the retina and the irradiation beam can be determined for all regions of the eye.

Alternatively, it is also possible to allow two different illumination beams 30 to reach the point 32 on the retina 16 via the same light path, but to vary the wavelength of the Irradiated light, for example, so that the same light path through the vitreous body is available for both beams, but, for example, in one case a wavelength is selected which corresponds to the optimum absorption wavelength of melanin on the retina, and in another case a wavelength which differs from this. This allows the pigmentation with melanin on the retina to be measured in a first approximation independently of inhomogeneities of the vitreous body of the eye if the wavelength-dependent attenuation of the vitreous body is neglected in a first step. Alternatively, the illumination beam can also have a broad spectrum or contain several wavelength ranges simultaneously. In this case, the sensor can differentiate over a wide wavelength range—even beyond the optically visible range—by means of wavelength-selective evaluation. For example, a hyperspectral sensor can be used for this purpose. Bayer filters can also be used to image the retina.

Combined measurements, in which both the light path and the wavelength are changed, can then also be used to determine the wavelength-dependent attenuation values of the vitreous body as a function of location. In this way, once the parameters of the light source 5′ are known, it is possible to take into account the attenuation of both the incident light or the incident radiation on the retina and the influence of the light path in the backscattered radiation, so that, for example, the pigmentation intensity with melanin on the retina can be determined objectively.

FIG. 5 schematically shows the structure of the linking of illumination parameters, measured values of the recording/camera image and the starting values for irradiation parameters. The illumination parameters 33 are designated x₁ to xₙ, wherein the individual parameters denote, for example, Illuminance levels, i.e. the radiant energy or power emitted by the illumination source, in each case for specific target regions, or also the distribution of the power over individual wavelength ranges of the emitted radiation, i.e. the shape of a wavelength spectrum with corresponding intensities. 35 refers to the mapping function or linking function, which can be designed as a self-learning algorithm, for example.

34 refers to the parameters y₁ to yₘ of the measured values captured by the camera 6 for a specific target region. If these are linked to the illumination parameters, this results in an n-tuple of starting values for irradiation parameters 36, which are designated z₁ to zₒ and which, for example, designate the size of the focal spot of the laser on the retina, the radiation intensity of the laser, the pulse duration and/or the number of pulses emitted for one or more target regions.

One goal of the invention is to determine optimized starting values for irradiation parameters for each target region. This result is initially achieved by determining the starting values, which is shown by the dashed dividing line 25 in FIG. 5. Actual laser operation of the laser 2 and thus treatment of the retina can then take place, and the influence of the laser treatment on the retina can then be carried out by repeating the process described, i.e. illuminating the retina with defined parameters and determining a camera image or recording. The result of the re-recorded camera image can be compared with the originally recorded camera image and a difference can be determined which shows the effect of the treatment by the laser 2. If the effect of the laser during treatment does not correspond to the treatment objective to be achieved, the algorithm 35 can be corrected or trained, and a new set of irradiation parameters can be determined from the illumination parameters during the second illumination and the measurement data recorded with the second camera image.

FIG. 6 first shows the sequence of process steps 20 to 24 as shown in FIG. 2, whereupon the treatment step 24 is followed by a further step 21′ of illuminating the retina with defined illumination parameters and simultaneously or offset recording of a camera image in a step 22′, whereupon the illumination parameters and the measurement data recorded with the camera image are combined in a further step 23′ to form new irradiation parameters.

FIG. 7 shows a flow chart in which the structure of the flow chart from FIG. 6 is repeated, wherein a further laser treatment 24′ follows the renewed linking 23′ of the illumination parameters and measurement data of the camera taken into account after a first laser effect 24. In addition, the information from the irradiation parameters determined in the correction process from step 23′ is used in a loop to improve the linking algorithm, which is performed in process steps 23 and 23′. This feedback from correction variables can be used to successively improve the linking algorithm on which the link between the illumination parameters, the camera measurement data and the starting values for irradiation parameters is based. For this purpose, treatments of one and the same individual or of groups of individuals, for example with a similar clinical picture, can be taken into account in order to improve the algorithm. The algorithm can also be stored centrally for this purpose, for example on a server or in a cloud, and retrieved for individual treatments, with correction variables then being fed back to the server or cloud in order to be able to use as much training data as possible from different treatment processes at different locations to train the algorithm.

FIG. 7 also indicates that in a further process step 37, additional parameters are measured on the individual patient or their eye or retina, which are fed into the linking step 23 for calibration. For this purpose, in step 37, for example, integral quantities of the retina, e.g. an average pigmentation or the strength of the coloration of the iris of the eye or the color of the iris or the strength of the skin pigmentation of the individual or the color of the skin pigmentation and/or the color and strength of the pigmentation of the hair of the individual can be measured with a sensor, in particular a camera, and fed into the linking step 23. The influence of these variables can also be taken into account when training the assignment algorithm or fed in as calibration parameters for individual treatments.

FIG. 8 schematically shows the development of the assignment algorithm A, A′ by linking corrected irradiation parameters 36′ with the original illumination parameters x₁ to xₙ and the measurement data y₁ to yₘ determined by the camera image. Before the individual training step, the starting values 36 for Irradiation parameters were assigned to the aforementioned input parameters, and the algorithm receives corresponding correction data 36′ with irradiation parameters z₁′ to zₒ′, which are interpreted as optimized result data and sharpen the algorithm.

This method optimizes the linking algorithm after the required number of training runs and thus also optimizes the starting values for irradiation parameters in a first step.

FIGS. 9, 10 and 11 show the functional relationship between the illuminance and the intensity of the reflected radiation for three different cases, in each case for the papilla/optic nerve and another location on the retina. The graph representing the papilla is labeled 50 in each case, while the graph representing a location in a target region of a treatment is labeled 51 in each case.

In principle, the method for determining the absorption intensity, which allows direct conclusions to be drawn about the pigmentation intensity of the retina, involves illuminating a location in the region of the papilla or at or on a blood vessel or at another special location on the retina, which is different from the target regions, with several different illuminances and simultaneously measuring the intensity of the reflected radiation. The functional relationship between the illuminance and the intensity of the reflected radiation is linear, resulting in an approximate straight line. At the very least, a straight line can be laid through the measuring points using linear regression, as shown in FIGS. 9, 10 and 11. The same procedure is carried out for one or more locations in the target regions of the retina that are to be treated later by radiation. The gradients of the straight lines determined represent the reflectivity at the various locations. Since the reflectivity in the region of blood vessels and at the papilla can be estimated well and is largely independent of other conditions of the individual retina, the values determined there can be used to calibrate the measured values of reflectivity values at the other locations. For this reason, the measured value of the intensity of the reflected radiation in the region of the papilla or the blood vessels can also be used to estimate the scattering and absorption losses of the radiation on the way to the respective location (papilla or blood vessels or another location where the reflectivity is known) and on the way back after reflection to the sensor with which the intensity of the reflected radiation is determined. This value of scattering and absorption can then also be assumed for the irradiation of the other regions of the retina, as it characterizes the light path through the vitreous body of the eye.

In detail, FIG. 9 shows an example in which the reflectivity and the absorption intensity in the macula region correspond to a standard case.

FIG. 10 shows a case in which the gradient of the straight line representing the macula is lower and therefore the reflectivity, measured against the gradient of the second straight line representing the papilla, is lower and therefore the absorption and pigmentation intensity is higher compared to the normal case.

FIG. 11 shows a case in which the slope of the straight line representing the macula is greater than the slope of the straight line representing the papilla in the cases shown in FIGS. 9 and 10. This measurement therefore represents the case of greater reflectivity at the macular location and correspondingly lower absorption intensity and pigmentation. Since the slope of the straight line 50 representing the measurement on the papilla is significantly lower than in the graphs of FIGS. 9 and 10, it can be concluded that, assuming the same reflectivity in the papilla region as in the cases of FIGS. 9 and 10, the absorption and/or scattering of the eye body is significantly stronger in the case shown in FIG. 11. An attenuation coefficient can be calculated from the differences in the slopes of the straight lines labeled 50 in FIGS. 9, 10 and 11, which represents the scattering and absorption in the case of the eye measured according to FIG. 11 and which can be taken into account in the irradiation during the treatment.

1. A method for determining irradiation parameters for [the] operation of a laser for irradiating one or more target regions of a retina of an eye of a patient using an irradiation beam, in which method the retina is fully or partly illuminated by an illumination device under defined illumination parameters, the method comprising:
during or shortly after illumination of the retina by the illumination device, capturing at least one optical recording of at least one target region of the retina; and
determining at least starting values of the irradiation parameters for irradiation of the at least one target region of the retina using the at least one optical recording.

2. The method according to claim 1, wherein at least one of an intensity of a pigmentation for one or more target regions of the retina or an intensity distribution of a pigmentation on the retina are determined using the at least one optical recording in order to determine the at least starting values of the irradiation parameters.

3. The method according to claim 1, wherein, in order to determine the at least starting values of the irradiation parameters for one or more target regions of the retina, one or more correction values are determined with respect to at least one of an expected absorption or scattering of a treatment beam in a vitreous body of the eye on the way to the one or more target regions to be irradiated, the one or more correction values being determined from an optical quality of the at least one optical recording, wherein the optical quality of the at least one optical recording includes at least one of an image sharpness or an image contrast.

4. The method according to claim 1, wherein, to produce a camera image, the retina is illuminated with radiation with a wavelength distribution, wherein the wavelength distribution includes at least one of infrared radiation or light in a wavelength range absorbed by melanin, wherein at least one of a luminous intensity of the illumination device, a total light power impinging on a unit area of the retina, or a light power impinging on a unit area of the retina is additionally determined in a defined wavelength range and wherein at least one of one or more brightness values or one or more colors of the camera image are used to determine the at least starting values of the irradiation parameters for the irradiation of the retina.

5. The method according to claim 1, wherein a thickness of the retina is determined for one or more target regions and is taken into account when determining the at least starting values of the irradiation parameters, wherein at least one of: i) the thickness of the retina is determined by creating an OCT thickness map of the retina, or ii) a type and intensity of a pigmentation of different regions of a body of the patient than the retina to be treated are used to determine the at least starting values of the irradiation parameters, wherein the type and intensity of the pigmentation of an iris of the eye or of skin or hair, are used to determine the at least starting values of the irradiation parameters.

6. The method according to claim 1, wherein one or more predetermined or specifiable reference recordings or reference images are used to determine the at least starting values of the irradiation parameters.

7. The method according to claim 1, wherein, in order to determine the irradiation parameters for one or more target regions of the retina to be irradiated, an intensity of a pigmentation of the at least one target region and an expected absorption of a treatment beam in an eye lens on the way to the at least one target region are determined independently of each other by carrying out at least two measurements under different illumination conditions, wherein at least one parameter is varied in the two or more measurements, wherein the at least one parameter includes: a size of a focused light spot on the retina, wherein in each case, a deviation from an expected light intensity distribution on an area of the focus spot is measured, a direction of incidence of an illumination beam of the illumination device through a pupil and a vitreous body for illumination of the respective target region, a direction of incidence to the at least one target region, a direction of emission of a detected reflected illumination beam of the illumination device, or at least one of a wavelength range or wavelength distribution of an illumination beam of the illumination device.

8. The method according to claim 1, wherein in order to determine the irradiation parameters for the at least one target region of the retina to be irradiated, at least one of an intensity of a pigmentation of the at least one target region or an expected absorption of a treatment beam in a lens of the eye on the way to the at least one target region are determined by carrying out at least two measurements, wherein a first measurement of the at least two measurements is directed to at least one of an absorption or a reflection characteristic in at least one of a region of a papilla or at least one blood vessel or part of a blood vessel in the retina, and wherein a second measurement of the at least two measurements is directed to at least one of an absorption characteristic or a reflection characteristic in a different region of the retina to be irradiated.

9. The method according to claim 1, wherein, in order to determine the irradiation parameters for one or more target regions of the retina to be irradiated, at least two locations of the retina are illuminated in each case successively or simultaneously with different illumination characteristics, wherein the different illumination characteristics include different illumination intensities, wherein, in each case, an intensity of a reflected signal is recorded, wherein for each of the at least two locations measured in this way, a functional relationship between the illumination characteristic and the intensity of the reflected signal is determined and that by comparing the functional relationships at the at least two measured locations, wherein comparing the functional relationships includes comparing at least one of slopes of function curves or a ratio of reflectivities at the at least two measured locations, and wherein a particular location of the at least two measured locations lies on a surface of a papilla or a blood vessel or at least partially comprises the surface of the blood vessel.

10. The method of claim 1, further comprising:
training an algorithm or a neural network for determining the at least starting values of irradiation parameters according to wherein, for a plurality of individual treatments, one or more datasets of at least one target region to be irradiated determined from one or more images from the at least one optical recording and one or more associated illumination parameters, wherein secondary information is additionally acquired in each case as input information, and wherein the irradiation parameters set during respective subsequent laser treatment as result variables are linked together as training data.

11. The Method according to claim 1, wherein, when determining the at least starting parameters for all target regions of the at least one target region, a calibration is carried out with one or more calibration parameters of the patient to be treated in each case, wherein the one or more calibration parameters include at least one of: a measured integral pigmentation intensity of skin of the patient, a measured integral pigmentation intensity of an iris of the eye of the patient, a measured pigmentation intensity of hair of the patient, a color of skin pigmentation of the patient, a color of the iris, or a color of the hair.

12. The method according to claim 1, wherein the at least one target region is irradiated with a titration beam or a treatment beam with the at least starting values of the irradiation parameters, wherein at least one continuation recording or at least one continuation camera image of the at least one target region of the retina irradiated with the treatment beam is then acquired, and wherein, using the at least one continuation recording or the at least one continuation camera image and the at least starting values of the irradiation parameters, the method further comprises:
determining at least continuation values of the irradiation parameters for the irradiation of the at least one target region of the retina.

13. An apparatus for irradiating one or more target regions of a retina of an eye with a treatment beam, the apparatus comprising:
a laser for generating the treatment beam;
an illumination device for illuminating at least one or more target regions of the retina with defined illumination parameters;
an optical recording device for capturing at least one optical recording or an image of at least one target region of the retina; and
a processing device configured to determine at least one or more initial values of one or more irradiation parameters for irradiating the one or more target regions of the retina with the treatment beam using the defined illumination parameters and the at least one optical recording or image.
